(19) United States
(12) Patent Application Publication
(10) Pub. No.: US 2024/0049816 A1
(43) Pub. Date: Feb. 15, 2024

(54) ELECTRIC FIELD PRODUCTION FOR REMEDIATION OF MICROORGANISMS

(71) Applicant: THE TRUSTEES OF INDIANA UNIVERSITY, Bloomington, IN (US)

(72) Inventors: Chandan K. SEN, Indianapolis, IN (US); Sashwati ROY, Indianapolis, IN (US); Subhadip GHATAK, Indianapolis, IN (US); Brent TOTO, Columbus, OH (US)

(21) Appl. No.: 18/259,457

(22) PCT Filed: Dec. 20, 2021
(86) PCT No.: PCT/US2021/064382

§ 371 Date: Jun. 27, 2023

Related U.S. Application Data
(60) Provisional application No. 63/132,837, filed on Dec. 31, 2020.

(51) Int. Cl. A41D 13/11; B01D 39/08; B01D 39/12; A61L 2/03

(52) U.S. Cl. CPC A41D 13/1192; B01D 39/08; B01D 39/12; A61L 2/03; A61L 2202/11; A61L 2202/14; A61L 2202/26; B01D 2239/0241; B01D 2239/0442; B01D 2239/0681

(57) ABSTRACT

This invention is directed to a multilayered fabric that comprises an electrode bearing surface that is electrically connected to a voltage source, wherein upon application of a charge, the electrode bearing will generating an electric field to neutralize pathogens and other microorganisms coming in contact with the material.

### CROSS REFERENCE TO RELATED APPLICATIONS

This application claims priority to the following: U.S. Provisional Patent Application No. 63/132,837 filed on Dec. 31, 2020, the disclosure of which is expressly incorporated herein.

### BACKGROUND OF THE DISCLOSURE

Protective fabrics can be incorporated into equipment used to provide safety to an individual from hazards. For example, personal protective equipment (PPE) pertains to equipment that, when worn by an individual, minimizes exposure to hazards that cause injury or infection. PPE can include face masks, helmets, googles, gowns, and other wearable equipment. Generally, PPE incorporates one or more protective fabrics to provide a barrier between the individual and the immediate environment. For instance, an individual may wear a face mask formed of a cloth fabric over his or her nose and mouth to mitigate transmission of infectious viruses to other individuals. Typically, during use, pathogens can make contact with and remain on the exterior surface regions of the mask. Consequently, transmission of such pathogens to the individual wearer can potentially occur as a result (e.g., if the wearer reuses the face mask without disinfecting the face mask beforehand, if the wearer touches the exterior surface regions, etc.).

### SUMMARY

In accordance with one embodiment a multilayered fabric is provided that comprises an electrode bearing surface that is electrically connected to a voltage source, wherein upon application of a charge the electrode bearing surface will generating an electric field to neutralize pathogens and other microorganisms coming in contact with the material. In accordance with one embodiment the voltage source is provided with a control for regulating the amount of charge dispensed to the electrode and thus regulating the strength of the electric field. In one embodiment the voltage source is a battery, optionally a rechargeable battery, that is electrically connected to the electrode bearing surface.

In accordance with one embodiment presented herein a battery-powered fabric design having electric field producing properties for neutralizing pathogens is disclosed. For example, the present disclosure may be embodied as personal protective equipment (PPE), such as a battery-powered multi-use face mask. The design thereof may provide a static or tunable electric field capable of remediating the infectivity, transmissibility, and or life of microorganisms (e.g., bacteria, archaea, protozoa, algae, fungi, viruses, and multicellular animal parasites (helminths)). In one embodiment the exterior surface of the mask carries a positive charge to neutralize the pathogens. In a further embodiment the positive charged exterior surface of the face mask is covered with a porous cloth or polymer matrix that will not impede contact of pathogens with generated electric field.

### BRIEF DESCRIPTION OF THE DRAWINGS

FIG. 1 illustrates a conceptual circuit diagram of an example PPE incorporating a fabric design having electric field producing characteristics, according to an embodiment.

FIG. 2 illustrates a conceptual circuit diagram of an example PPE incorporating a fabric design having electric field producing characteristics, according to an embodiment.

FIG. 3 illustrates exploded and perspective views of an example PPE incorporating a fabric design having electric field producing characteristics in a three-layer electrode pattern configuration, according to an embodiment.

FIG. 4 illustrates exploded and perspective views of an example PPE incorporating a fabric design having electric field producing characteristics in a three-layer electrode mesh configuration, according to an embodiment.

FIG. 5 illustrates exploded and perspective views of an example PPE incorporating a fabric design having electric field producing characteristics in an interdigitated conductive pattern, according to an embodiment.

FIG. 6 illustrates various views of an example PPE incorporating a fabric design having electric field producing characteristics in a multi-layer through-hole configuration, according to an embodiment.

FIG. 7 illustrates exploded and perspective views of an example PPE incorporating a fabric design having electric field producing characteristics in an interdigitated electrode pattern with a conductive coating, according to an embodiment.

FIG. 8 illustrates exploded and perspective views of an example PPE incorporating a fabric design having electric field producing characteristics in an interdigitated electrode pattern with an external conductive material, according to an embodiment.

FIG. 9 illustrates a perspective view of an example layer of PPE incorporating a fabric design having electric field producing characteristics having a continuous strap, according to an embodiment.

FIG. 10 illustrates a perspective rear view of an example PPE incorporating a fabric design having electric field producing characteristics having a continuous wraparound strap, according to an embodiment, optionally wherein the wraparound strap is provided with means for adjusting the length of the wraparound strap.

### DETAILED DESCRIPTION

### Definitions

In describing and claiming the invention, the following terminology will be used in accordance with the definitions set forth below.

As used herein, the term “treating” includes alleviation of the symptoms associated with a specific disorder or condition and/or preventing or eliminating said symptoms. “Inhibition” of disease progression or disease complication in a subject means preventing or reducing the disease progression and/or disease complication in the subject.

As used herein an “effective” amount or a “therapeutically effective amount” refers to a nontoxic but sufficient amount of a therapeutic treatment to provide the desired effect. An appropriate “effective” amount in any individual case may be determined by one of ordinary skill in the art using routine experimentation.

As used herein the term “patient” without further designation is intended to encompass any warm blooded vertebrate domesticated animal (including for example, but not limited to livestock, horses, mice, cats, dogs and other pets) and humans receiving a therapeutic treatment whether or not under the supervision of a physician.

“Subject” can refer to living organisms such as mammals, including, but not limited to humans, livestock, dogs, cats, and other mammals.

### Embodiments

Embodiments presented herein disclose a fabric design having electrical field producing characteristics that neutralize pathogens (e.g., bacteria, archaea, protozoa, algae, fungi, viruses, and multicellular animal parasites (helminths)) upon contact. Preparation of personal protective equipment (PPE) comprising such materials can be used to limit the infectivity, transmissibility, and viaility of pathogenic organism, and thus minimize adverse health effects on humans or animals caused by the pathogen. The various embodiments disclosed herein employ the principle of neutralizing pathogen infectivity through the use of a positively charged surface. For example, embodiments may be adapted to personal protective equipment (PPE), such as a wearable face mask having electrode layers. One of the layers, such as an exterior surface layer, includes positive electrodes to neutralize the pathogens on contact. As further described herein, a variety of electrode and conductive material configurations for the fabric design (described relative to the face mask) may be used to provide the layers used to generate the electrical field.

In accordance with one embodiment, the present disclosure uses a face mask having electrical field producing characteristics as a reference example of an implementation of a fabric design that uses electrical fields to neutralize potentially harmful pathogens. However, one of skill in the art will recognize that embodiments presented herein may be adapted to a variety of applications, such as PPE and other medical equipment. For example, embodiments may be adapted to medical or surgical gowns. The gown may be configured with one or more electrode layers and/or conductive material used to generate an electrical field for neutralizing the transmission of pathogens to or from the wearer. As another example, embodiments may be adapted to an external or internal wound dressing that is configured with one or more electrode layers and/or conductive material used to generate an electrical field for neutralizing the transmission of pathogens to or from the wearer. Doing so may provide increased healing effects and allow biofilm material to be susceptible to antibiotics. As yet another example, embodiments may be adapted to a passenger seat (e.g., in an airplane, bus, or other public transportation). The passenger seat may have one or more conductive layers producing an electric field on the exterior surface of the seat to disinfect the seat, e.g., between use. As yet another example, embodiments may be adapted to a medical sheath applied topically on an individual to inactivating viruses transmitted via contact with human skin, e.g., herpes simplex virus, shingles, etc.

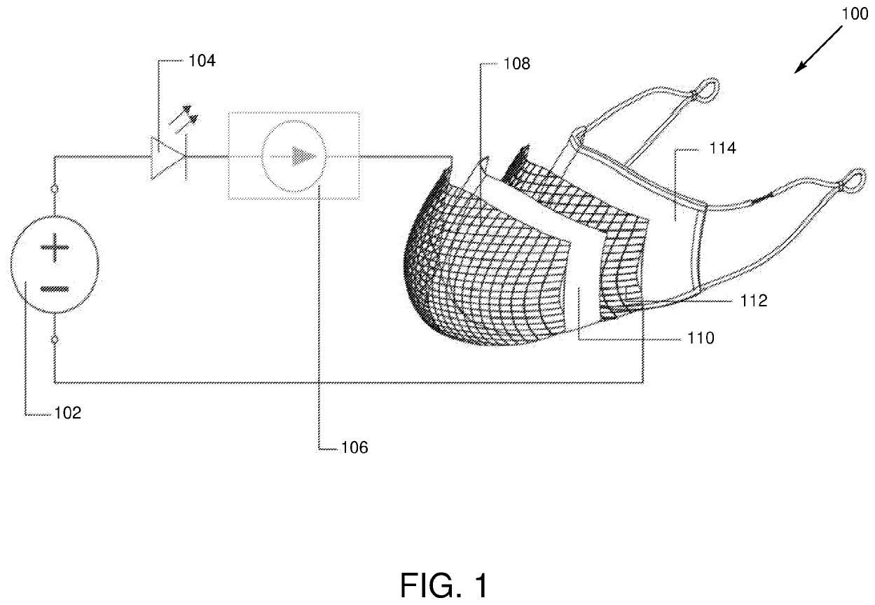

Referring now to FIG. 1, a conceptual diagram of a face mask 100 incorporating a fabric design having electric field generating properties is shown. Illustratively, the face mask 100 comprises a core mask 114, which may be of various materials, such as cloth, non-woven fibers, etc. The core mask 114 provides a covering or filter for the nose and mouth. A strap is engaged with peripheral portions of the face mask 100 to fasten the face mask 100 to a face of an individual wearer. Further, the face mask 100 includes a number of filter layers (e.g., the fabric design) to be placed atop the core mask 114. As shown, the layers include a positive electrode layer 108, a conductive material layer 110, and a negative electrode layer 112. The layers may be secured to the core mask 114 through a variety of methods, including but not limited to using an adhesive to engage each layer, stitching the layers together, using non-conductive fasteners or using magnets to hold the layers together. The positive electrode layer 108 provides an exterior surface for the face mask 100. The electrode layer 108, which is exposed to the conductive material layer 110 (e.g., formed of a copper material, of a conductive gel, etc.) may be charged via a voltage source 102 (e.g., a 6 vDC battery), generating an electric field, thereby enabling the face mask 100 to neutralize pathogens and other microorganisms. The voltage source 102 may be a disposable or rechargeable source.

Further, the fabric design may enable a user of the face mask 100 (e.g., a wearer) to tune the charge provided by the voltage source 102. Doing so may increase the effectiveness of neutralizing certain pathogens. More particularly, different microorganisms respond differently to different electric field strengths. For example, a charge producing a high electric field may inactivate certain viruses (e.g., the coronavirus causing COVID-19) more effectively than a lower electric field. By tuning the charge to create a higher electric field, such viruses may be more quickly inactivated. As another example, if the fabric design is incorporated into a wound dressing, initially generating a high electric field for the dressing (e.g., at 6V) and then gradually lowering the electric field (e.g., to 3V) may increase the rate at which the underlying wound heals.

In addition, the positive electrode layer 108 may include one or more light emitting diodes (LEDs) 104, which, when activated, indicate that the face mask 100 is in a powered mode. In an embodiment, a constant current regulator 106 may be configured within the face mask 100 to regulate current to the positive electrode layer 108. For example, the constant current regulator 106 may be embodied as a 10 mA current limiter.

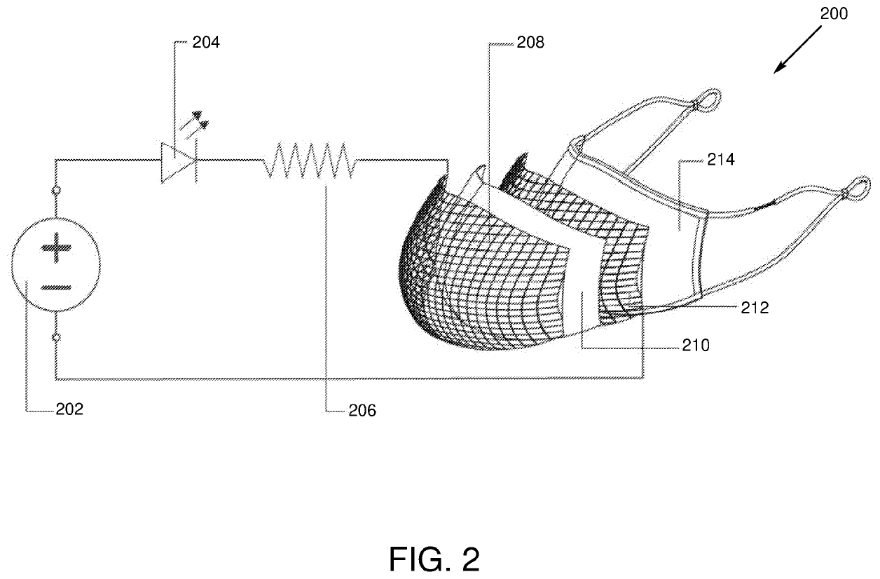

Referring now to FIG. 2, a conceptual diagram of a face mask 200 incorporating a fabric design having electric field generating properties is shown. The face mask 200 includes a voltage source 202, LEDs 204, positive electrode layer 208, conductive material layer 210, negative electrode layer 212, and core mask 214, which may be identical to components 102, 104, 108, 110, 112, and 114 of FIG. 1, respectively. In an embodiment, the face mask 200, in place of a constant current regulator, may use a resistor 206 to regulate voltage from the voltage source 202 to the positive electrode layer 208.

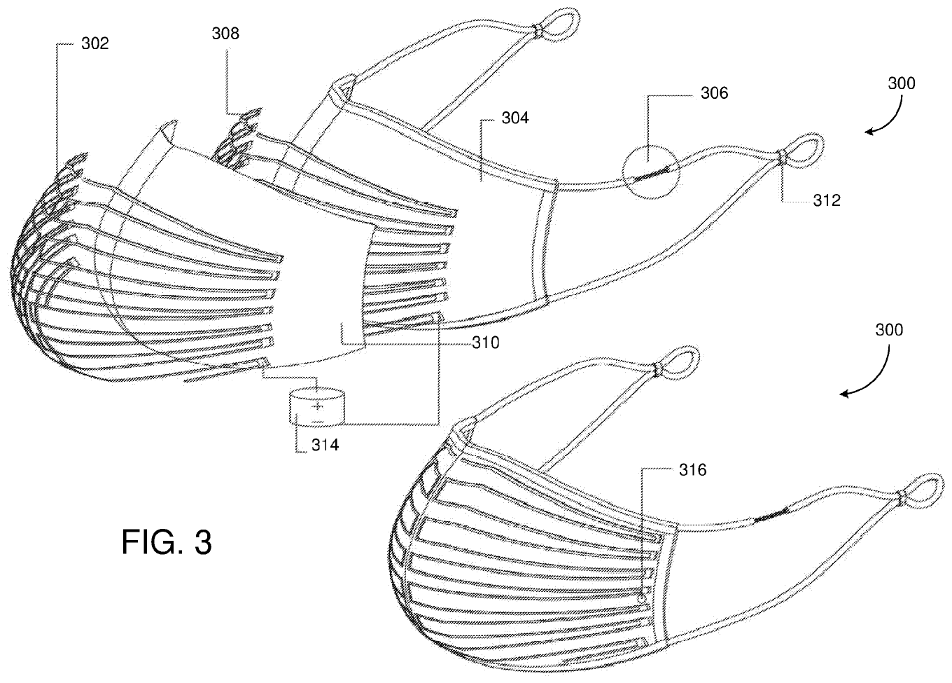

Referring now to FIG. 3, an embodiment of the face mask incorporating a fabric design having electric field generating properties is shown, in which a face mask 300 is configured in a three-layer electrode pattern. The upper portion of FIG. 3 shows the face mask 300 in an exploded view. As shown, the face mask 300 includes a positive electrode layer 302, a core mask 304, mask strap electrodes 306, a negative electrode layer 308, a conductive material layer 310, a strap adjuster 312, and a voltage source 314. The electrodes forming the layers 302 and 308 may be printed on the conductive material layer 310, printed on independent layers, or may be independent electrodes. The electrodes 306 allow neutralization of pathogens on the surface of the mask strap. The bottom portion of FIG. 3 shows the face mask 300 in a perspective view. As can be seen in this view, the face mask 300 includes a LED conductivity indicator 316 to indicate that the face mask 300 is powered on (via the voltage source 314). In one embodiment the mask strap comprises an elastic material.

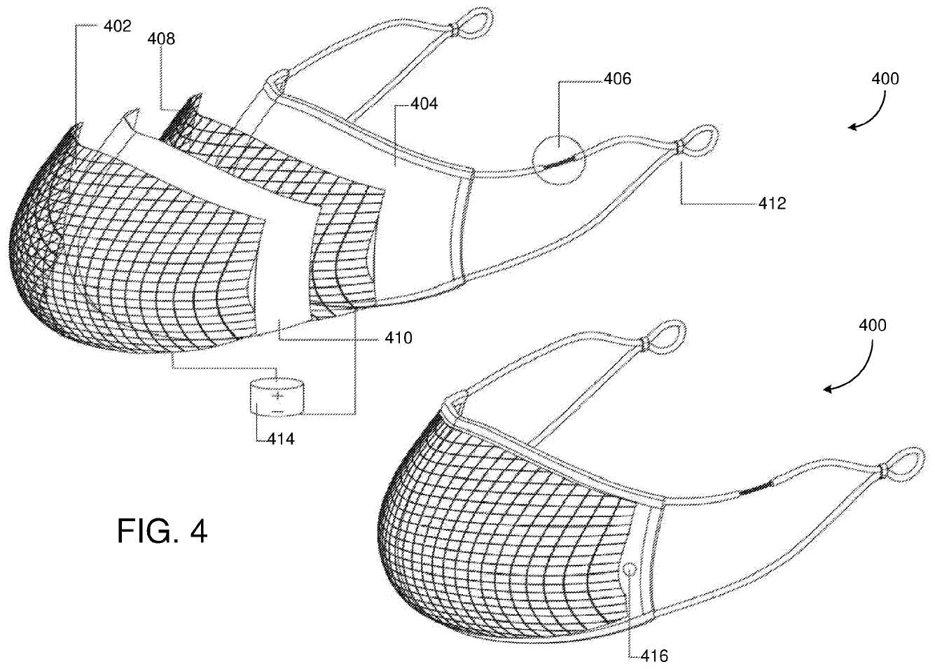

Referring now to FIG. 4, an embodiment of the face mask incorporating a fabric design having electric field generating properties is shown, in which a face mask 400 is configured in a mesh pattern. The upper portion of FIG. 4 shows the face mask 400 in an exploded view, and the bottom portion of FIG. 4 shows the face mask 400 in a perspective view. As shown, the face mask 400 includes a positive electrode layer 402, a core mask 404, mask strap electrodes 406, a negative electrode layer 408, a conductive material layer 410, a strap adjuster 412, and a voltage source 414. Illustratively, the electrodes forming the layers 402 and 408 form a mesh pattern. The electrodes forming the layers 402 and 408 may be printed on the conductive material layer 410, printed on independent layers, or be independent mesh material. As can be seen in the perspective view, the face mask 400 includes a LED conductivity indicator 416 to indicate that the face mask 400 is powered on (via the voltage source 414).

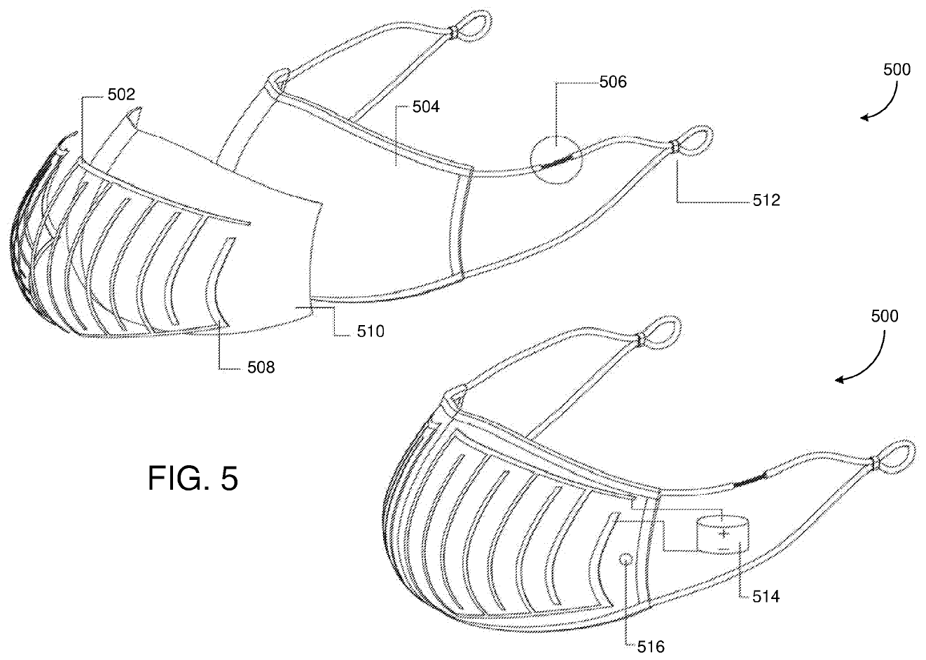

Referring now to FIG. 5, an embodiment of the face mask incorporating a fabric design having electric field generating properties is shown, in which a face mask 500 is configured in an interdigitated conductive pattern. The upper portion of FIG. 5 shows the face mask 500 in an exploded view, and the bottom portion of FIG. 5 shows the face mask 500 in a perspective view. The interdigitated conductive pattern includes a layer of a positive electrode 502 and a layer of a negative electrode 508 in an interdigitated arrangement relative to one another. The layers may be placed or printed over a conductive material layer 510, which is placed over a core mask 504. The layer created by the electrodes 502 and 508 provide an exterior surface on which an electrical field is generated to neutralize pathogens. The face mask 500 also includes mask strap electrodes 506, an adjuster 512, a voltage source 514, and a LED conductivity indicator 516 (similar to components 506, 512, 514, and 516, respectively).

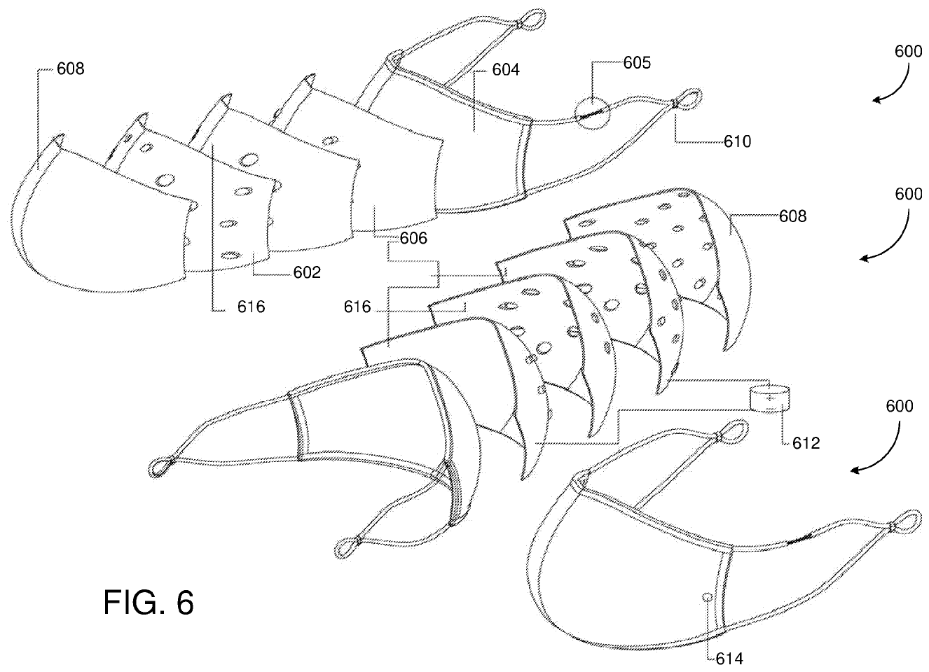

Referring now to FIG. 6, an embodiment of the face mask incorporating a fabric design having electric field generating properties is shown, in which a face mask 600 is configured using through-hole layers. FIG. 6 depicts the face mask 600 in a front exploded view (top), rear exploded view (middle), and perspective view (bottom). As shown, the face mask 600 includes a positive electrode layer 602, core mask 604, mask strap electrodes 605, a negative electrode layer 606, a conductive material layer 608, an adjuster 610, a voltage source 612, a LED conductivity indicator 614, and an insulating material 616 (e.g., a neoprene material, rubber material, foam material, etc.). In this configuration, the conductive material layer 608 provides an exterior surface for the face mask 600, which, when activated, neutralizes pathogens on contact.

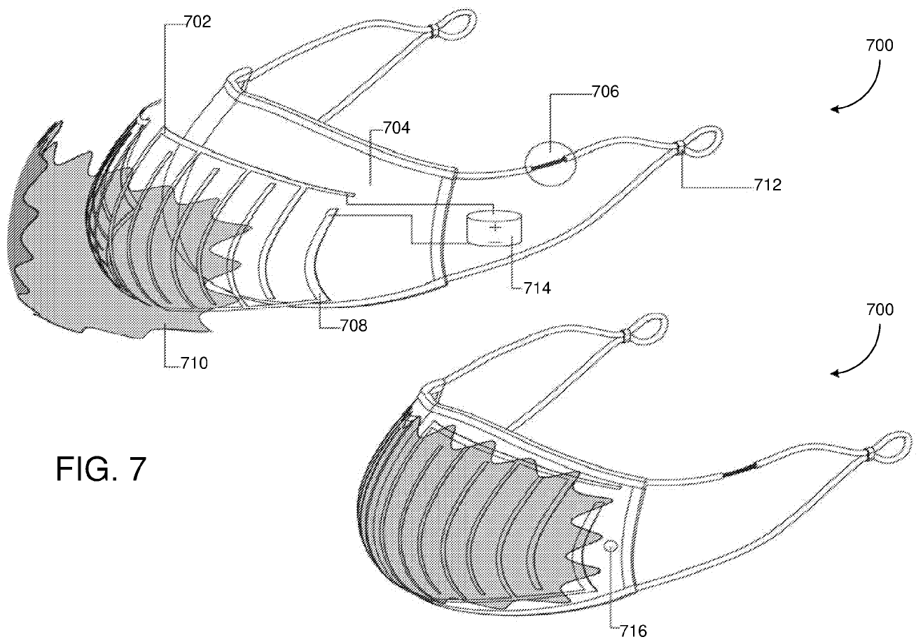

Referring now to FIG. 7, an embodiment of the face mask incorporating a fabric design having electric field generating properties is shown, in which a face mask 700 is configured with a conductive coating 710 as an exterior surface for a core mask 704. FIG. 7 depicts the face mask 700 in an exploded view (top) and a perspective view (bottom). As shown, the conductive coating 710 is layered atop a layer of positive electrodes 702 and negative electrodes 708. The electrode layer is layered atop a core mask 704. The face mask 700 includes mask strap electrodes 706, an adjuster 712, a voltage source 714, and a LED conductivity indicator 716, which are similar to their respective components in the previously described figures.

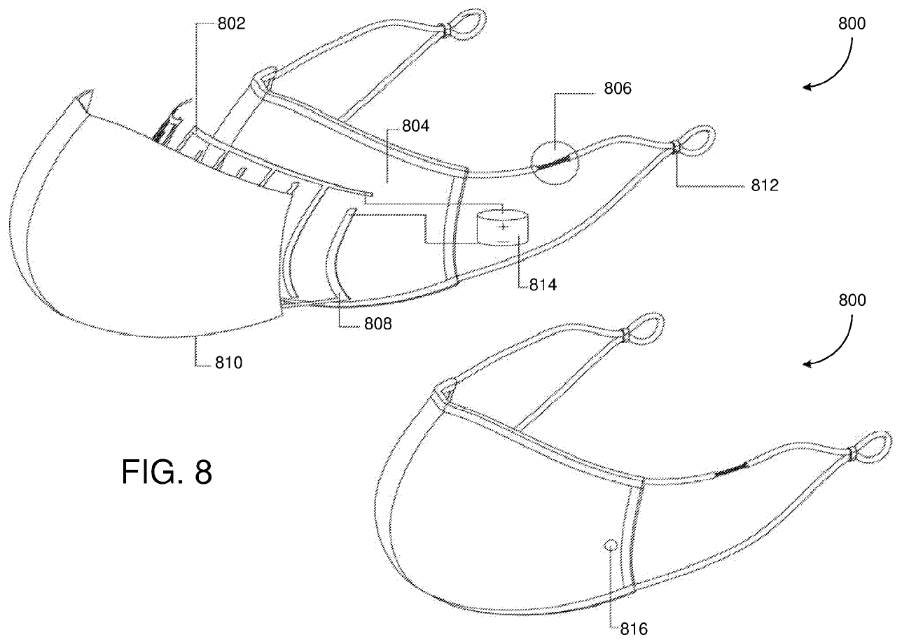

Referring now to FIG. 8, an embodiment of the face mask incorporating a fabric design having electric field generating properties is shown, in which a face mask 800 is configured with an exterior conductive material 810 over an interdigitated pattern of positive electrodes 802 and negative electrodes 808. These layers may be placed atop a core mask 804. The exterior conductive material 810 provides an exterior surface area for the face mask 800, which when activated, neutralizes pathogens on contact. The face mask 700 also includes mask strap electrodes 806, an adjuster 812, a voltage source 814, and a LED conductivity indicator 816, which are similar to their respective components in the previously described figures.

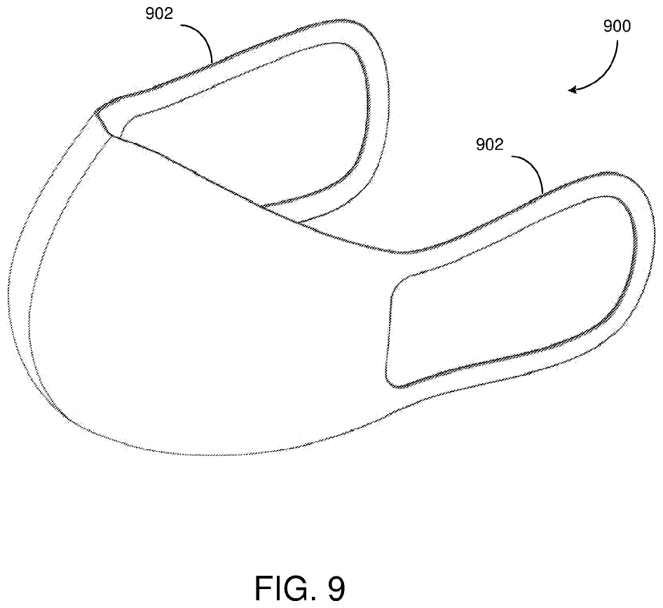

Referring now to FIG. 9, an embodiment of a layer 900 of a fabric design having electric field generating properties is shown, in which the layer 900 incorporates embodiments described herein. The layer 900 may correspond to any of the conductive material layer, positive electrode layer, negative electrode layer, and core mask described herein. In this embodiment, the layer 900 includes straps 902 that extend continuously from peripheral ends from the front, allowing the resulting mask to wrap around ears of an individual wearer. Doing so allows the extension from the layers to also possess electric field generating characteristics to neutralize pathogens

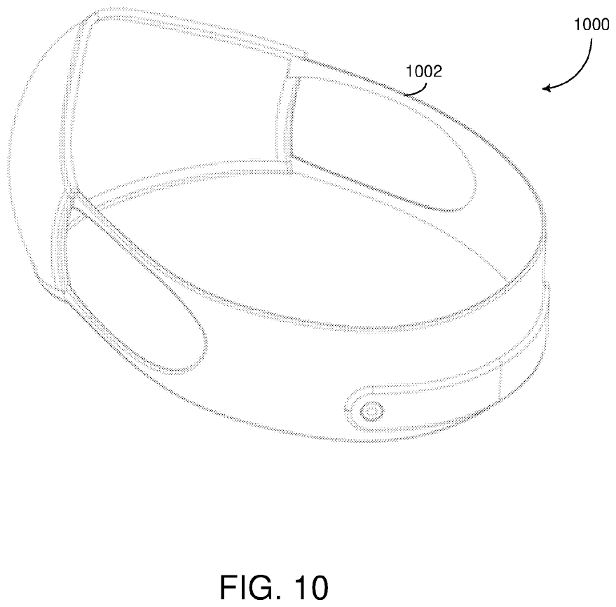

Referring now to FIG. 10, an embodiment of the face mask incorporating a fabric design having electric field generating properties is shown, in which layers 1002 of the face mask 1000 (e.g., a core mask layer, a conductive material layer, a positive electrode layer, and a negative electrode layer) extend in a wraparound fashion from peripheral ends from the front, such that the face mask 1000 wraps around the head of an individual wearer. Doing so allows the extension from the layers to also possess electric field generating characteristics to neutralize pathogens.

While the disclosure has been illustrated and described in detail in the drawings and foregoing description, such an illustration and description is to be considered as exemplary and not restrictive in character, it being understood that only illustrative embodiments have been shown and described and that all changes and modifications that come within the spirit of the disclosure are desired to be protected. A plurality of advantages of the present disclosure arise from the various features of the method, computer-readable storage medium, and system described herein. It will be noted that alternative embodiments of the method, apparatus, and system of the present disclosure may not include all of the features described yet still benefit from at least some of the advantages of such features. Those of ordinary skill in the art may readily devise their own implementations of the method, apparatus, and system that incorporate one or more of the features of the present invention and fall within the spirit and scope of the present disclosure as defined by the appended claims.

In accordance with embodiment 1 an antimicrobial fabric is provided that inhibits the infectivity and/or viability of pathogenic organisms that come in contact with the fabric. The fabric can be used to prepare personal protective equipment (PPE), including garments and masks, to limit the infectivity, transmissibility, and viability of pathogenic organisms that come in close proximity to the fabric. Products comprising such a protective fabric can be prepared, including PPE, to minimize adverse health effects on humans or animals caused by the pathogen. In accordance with embodiment 1 the protective antimicrobial fabric comprises at least one first layer comprising a composition that can be positively charged, optionally wherein the composition is electrically connected to a voltage source, wherein the voltage source, when activated, causes the first layer to be positively charged.

In accordance with embodiment 2 the protective fabric of embodiment 1 is provided, wherein the composition of the first layer is a conductive material.

In accordance with embodiment 3 the protective fabric of embodiment 1 or 2 is provided, wherein the conductive material is a conductive gel.

In accordance with embodiment 4 the protective fabric of any one of embodiments 1-3 is provided, wherein the protective fabric is incorporated as part of a face mask.

In accordance with embodiment 5 the protective fabric of any one of embodiments 1˜4 is provided, wherein the protective fabric comprises a second layer and an intermediate layer, wherein
said first layer comprises a positive electrode;
said second layer comprises a negative electrode; and
said intermediate layer comprises a conductive material wherein the first and second layer is attached on opposing sides of the intermediate layer.

In accordance with embodiment 6 the protective fabric of embodiment 5 is provided, wherein the first and second layers are each formed as a mesh.

In accordance with embodiment 7 the protective fabric of any one of embodiments 1˜4 is provided, wherein the protective fabric further comprises a conductive material layer, wherein the first layer comprises a positive electrode and a negative electrode formed in an interdigitated conductive pattern on the surface of the conductive material layer. In one embodiment the protective fabric is formed using various materials, such as cloth, non-woven fibers, etc.

In accordance with embodiment 8 the protective fabric of any one of embodiments 1-7 is provided, wherein at least one of the one or more layers is in a through-hole pattern.

In accordance with embodiment 9 the protective fabric of any one of embodiments 1-8 is provided, wherein the voltage source provides a charge that is tunable.

In accordance with embodiment 10 the protective fabric of embodiment 9 is provided, wherein the charge is set at a level that inactivates a pathogen coming in direct contact and/or close proximity with said positively charged first layer.

In accordance with embodiment 11 the protective fabric of any one of embodiments 1-10 is provided, wherein the one or more layers extend from peripheral ends of a front portion of the protective fabric.

In accordance with embodiment 12 the protective fabric of any one of embodiments 1-11 is provided, wherein the one or more layers extend in a wraparound fashion.

In accordance with embodiment 13 a face mask is provided comprising
a core mask configured to have an interior surface shaped as a covering for the nose and mouth, an exterior surface formed to receive a filter layer, and having a left peripheral portion and a right peripheral portion;
a strap having a first and second end, wherein the first end is attached to said left peripheral portion and the second end is attached to said right peripheral portion;
a filter layer secured to the core mask, wherein said filter layer comprises
   a positive electrode located on an exterior surface of said filter layer;
   a negative electrode; and
   a voltage source that is in electrical communication with said positive electrode and negative electrode.

In accordance with embodiment 14 the face mask of embodiment 13 is provided wherein the filter further comprises a conductive material layer, wherein the conductive material layer is fixed between said positive electrode and the negative electrode.

In accordance with embodiment 15 the face mask of embodiment 13 or 14 is provided wherein the positive and negative electrodes are each formed in a mesh pattern.

In accordance with embodiment 16 the face mask of embodiment 13 is provided wherein the positive and negative electrodes are located on the same surface and formed in an interdigitated conductive pattern.

In accordance with embodiment 17 the face mask of embodiment 16 is provided wherein the positive and negative electrodes are located on the surface of a conductive material and said conductive material is attached to the exterior surface of the core mask.

In accordance with embodiment 18 the face mask of embodiment 17 or 18 wherein a porous conductive coating is layered on top of said positive and negative electrodes. In accordance with one embodiment the electrode layers of the mask (e.g., formed of a copper material, of a conductive gel, etc.) may be charged via a voltage source (e.g., a 6 vDC battery), generating an electric field, thereby enabling the face mask to neutralize pathogens and other microorganisms that come in contact or close proximity with the protective material or mask. The voltage source may be a disposable or rechargeable source.

The layers may be secured to each other and to the core mask through a variety of methods, including but not limited to using an adhesive to engage each layer, stitching the layers together, using non-conductive fasteners or using magnets to hold the layers together.

1. A protective fabric comprising:
one or more layers, with a first layer being an exterior surface; and
a voltage source, which, when activated, causes the first layer to be positively charged.

2. The protective fabric of claim 1, wherein the first layer is a conductive material.

3. The protective fabric of claim 2, wherein the conductive material is a conductive gel.

4. The protective fabric of claim 1, wherein the protective fabric is incorporated as part of a face mask.

5. The protective fabric of claim 1, wherein the protective fabric comprises a second layer and an intermediate layer, wherein
said first layer comprises a positive electrode;
said second layer comprises a negative electrode; and
said intermediate layer comprises a conductive material wherein the first and second layers are attached on opposing sides of the intermediate layer.

6. The protective fabric of claim 5, wherein the first and second layers are each formed as a mesh.

7. The protective fabric of claim 1, wherein the protective fabric further comprises a conductive material layer, wherein the first layer comprises a positive electrode and a negative electrode formed in an interdigitated conductive pattern on the surface of the conductive material layer.

8. The protective fabric of claim 1, wherein at least one of the one or more layers is in a through-hole pattern.

9. The protective fabric of claim 1, wherein the voltage source provides a charge that is tunable.

10. The protective fabric of claim 9, wherein the charge is set at a level that inactivates a pathogen coming in direct contact with said positively charged first layer.

11. The protective fabric of claim 1, wherein the one or more layers extend from peripheral ends of a front portion of the protective fabric.

12. The protective fabric of claim 11, wherein the one or more layers extend in a wraparound fashion.

13. A face mask comprising
a core mask configured to form a covering for the nose and mouth and having a left peripheral portion and a right peripheral portion;
a strap having a first and second end, wherein the first end is attached to said left peripheral portion and the second end is attached to said right peripheral portion;
a filter layer secured to the core mask, wherein said filter layer comprises
a positive electrode located on an exterior surface of said face mask;
a negative electrode; and
a voltage source that is in electrical communication with said positive electrode and negative electrode.

14. The face mask of claim 13 wherein the filter further comprises
a conductive material layer, wherein the conductive material layer is sandwiched between said positive electrode and the negative electrode.

15. The mask of claim 14 wherein the positive and negative electrodes are each formed in a mesh pattern.

16. The mask of claim 13 wherein the positive and negative electrodes are located on the same surface and formed in an interdigitated conductive pattern.

17. The mask of claim 16 wherein the positive and negative electrodes are located on the surface of a conductive material and said conductive material is attached to the exterior surface of the core mask.

18. The mask of claim 17 wherein a conductive coating is layered on top of said positive and negative electrodes.
